(19) United States
(12) Patent Application Publication
(10) Pub. No.: US 2006/0228301 A1
(43) Pub. Date: Oct. 12, 2006

(54) Methods and compositions for determining targeted drug sensitivity and resistance in a cancer subject

(76) Inventor: Laszlo Boros, Los Angeles, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/100,069

(22) Filed: Apr. 6, 2005

(51) Int. Cl. A61K 49/00; A61K 31/70

(52) U.S. Cl. 424/9.1; 514/23

(57) ABSTRACT

Diagnostic and therapeutic methods of cancer treatment and prevention using metabolic profiling compounds that contain [1,2-¹³C₂]-D-glucose, and kits for using such metabolic profiling compounds.

### FIELD OF THE INVENTION

This invention relates generally to the field of biochemical methodologies. The invention further relates to the field of stable ¹³C-isotope labeling of nucleic acid synthesis precursors to examine changes in metabolic pathways incident to cancer and drug resistance in cancer.

### BACKGROUND OF THE INVENTION

Drug development against cancer in recent years is becoming more focused on and targeted against narrow gene and protein constructs expressed primarily in tumor cells. This approach is called the targeted era of drug design and requires set of validated target genes and proteins to inhibit growth signaling pathways. Although this approach offers less toxicity and more specificity against individual tumor types, a significant limitation of targeted drugs is their narrow, target-dependent action on a metabolic network, which inherently possesses a hierarchy of metabolic reactions for alternative macromolecule synthesis processes within the metabolic network.

Metabolic profiling or metabolomics is an old investigative field where the amounts or concentrations of various metabolites of various pathways in living organisms are measured and, from these determinations, activities of the respective metabolic pathways are predicted (Katz, J., Rognstad, R., Biochemistry 6: 2227-47 (1967)). Specific examples include the labeling of pentose phosphate from glucose-¹⁴C and estimation of the rates of transaldolase, transketolase, and their contribution to ribose phosphate synthesis in the pentose cycle. In general, these techniques only provide information on a static picture of a healthy cultured cell at one point in time and only measure synthesis rates without being able to reveal specific reactions and their contributions to end-product synthesis. The technique does not exactly reveal the previous metabolic steps and the exact synthesis pathways but only estimates the involvements of possible metabolic pathways based on existing biochemical information. Model systems based on classic metabolomics are severely outdated and incorrect as they estimate fluxes based on assumptions and predictions that do not hold using modern techniques.

There are many alternative pathways throughout cellular metabolism to produce various metabolites, which may make it difficult to elucidate particular enzymatic reactions using static metabolic profiling. Measuring metabolite levels generally does not reveal substrate flow and enzymatic substrate modifications in interconnected and complex metabolite networks, where alternative synthesis routes are common and may be prominent. The problem is known as the hidden phenotype of a particular metabolic defect, where metabolites are still produced but via alternative pathways. (Raamsdonk, L. M., et al. (2001). Nat Biotechnol 19: 45-50).

Leading laboratories in stable isotope based metabolite research use single labeling patterns and measure single pathways in mammalian cells in order to reveal specific synthesis steps of bio-molecules. These pathways may be involved in cell proliferation (Neese, R. A., et al. (2001). Anal. Biochem. 298: 189-95). However these methods generally measure new cell production through DNA synthesis without the specifics of metabolic pathway activities and their contribution to the cellular proliferation process (Turner, S. Curr Opin Drug Discov Devel. 2005 January; 8(1):115-26). Further, others have carried out work applied to gluconeogenesis, as well as de novo lipid and fatty acid synthesis. (See, Previs, S. F., et al. (1998) Curr Opin Clin Nutr Metab Care 1: 461-5).

Stable isotope studies of phytanic acid alpha-oxidation and in vivo production of formic acid has also been described (Eur J Pediatr 56: S83-7). Stable isotopes are also used as standards for quantification of known compounds in the blood and body fluids and others have described stable isotope dilution negative ion chemical ionization gas chromatography-mass spectrometry for the quantitative analysis of paroxetine in human plasma as well as the clinical measurement of steroid metabolism. Although important for the quantitation of metabolite synthesis and turnover rates, these papers do not identify, analyze or determine relative contributions from alternative pathways of producing metabolites or apply such analysis to predictive medicine or drug effects. In particular, drug resistance and unforeseen side effects are increasing problems in targeted drug design, medicine and clinical research, and have not been heretofore investigated using an approach that measures differential generation of metabolites via alternate routes to determine clinical stages of drug resistance. This is particularly true in the era of targeted drugs, when compounds are narrowly aimed against genetic or protein targets which exert strong control on certain downstream metabolic pathways but lack control on alternative synthesis routes, which inherently exist and thus escape drug effect. Applying the basic principle that each targeted drug is only as potent as the target itself in controlling metabolic pathways, it is clear that drugs selective against narrow genetic or proteomic targets posses selectivity but also severe limitations in metabolite flow control in the highly complex and interconnected channels of enzymatic reactions and molecule synthesis.

### SUMMARY OF THE INVENTION

The present invention provides novel diagnostic and therapeutic methods for use in mammalian subjects suffering from cancer. In particular, it has been found that compounds containing or derived from [1,2-³C₂]-D-glucose are useful to measure metabolic profile changes, particularly nucleic acid synthesis, associated with resistance to conventional cancer therapeutics or new targeted drugs. [1,2-¹³C₂]-D-glucose produces [1-¹³C₁]-D-ribose or [1-¹³C₁]-D-deoxyribose (also known as m1) in the oxidative branch of the pentose cycle of nucleic acid synthesis, and produces [1,2-¹³C₂]-D-ribose or [1,2-¹³C₂]-D-deoxyribose (also known as m2) via the non-oxidative branch of the pentose cycle. The oxidative and non-oxidative branches of the pentose cycle constitute alternative synthesis pathways of the same sugar phosphate, ribose-5-phosphate, which is the inherently preserved backbone sugar of all ribo-nucleic and deoxyribo-nucleic acids in all species.

In one aspect of the invention, the invention provides a method of determining the likelihood of a subject's reduced response to treatment with a cancer therapeutic by administering to the subject a metabolic profiling compound that contains [1,2-¹³C₂]-D-glucose, obtaining from the subject a biological sample, and determining the ratio of [1-¹³C₁]-D-ribose to [1,2-¹³C₂]-D-ribose in the biological sample [(m1/Σm)/(m2/Σm)]. Generally, a ratio of 1 (i.e., 1:1) indicates that the subject has glucose flow toward nucleic acid synthesis equally balanced via the oxidative and non-oxidative branches of the pentose cycle. Any change (e.g., an increase or a decrease) in the ratio indicates that the subject has developed or may develop altered response to a cancer therapeutic, or is at risk of having a reduced response to treatment with the cancer therapeutic. Determining this ratio before and after a treatment provides highly significant reference points when a response to targeted drugs is measured. As genetic and protein drug targets generally only control either major branch of pentose phosphate synthesis in the cycle, early drug resistance and failure can be identified by determining small but consistent changes in the ratio of oxidative/non-oxidative ribose synthesis. In other words, if the ratio is higher than about one, fractional ribose synthesis in the oxidative branch is increased and vice versa. A ratio less than about 1 indicates increased non-oxidative ribose synthesis and more aggressive tumor formation or growth. In some embodiments of the invention, a ratio of 0.5 or lower indicates that the subject is or is at risk of becoming completely unresponsive to treatment with the cancer therapeutic. In embodiments of the invention, the cancer therapeutic is a tyrosine kinase inhibitor, such as imatinib (Gleevec™). In embodiments, the subject suffers from chronic myeloid leukemia (CML; also known as chronic myelogenous leukemia) or a gastrointestinal stromal tumor (GIST).

The invention provides that the biological sample obtained from the subject can be blood, a tumor biopsy, a tumor aspirate, a cultured tumor cell, or bone marrow. The invention also provides that the step of determining the ratio of [1-¹³C₁]-D-ribose to [1,2-¹³C₂]-D-ribose in the obtained sample is performed using gas chromatography-mass spectroscopy (GC-MS) or nuclear magnetic resonance (NMR). The invention further provides that the step of determining the ratio of [1-¹³C₁]-D-ribose to [1,2-¹³C₂]-D-ribose is performed using peak integration and imaging of tumors using magnetic resonance imaging (MRI) by frequencies corresponding to the [1-¹³C₁]-D-ribose and [1,2-¹³C₂]-D-ribose isotopomers in situ.

In another aspect, the invention provides a method of selecting an appropriate therapeutic for treatment in a patient suffering from cancer who is partially or fully non-responsive to tyrosine kinase inhibitory treatment, by obtaining from the patient one or more tumor cells, culturing the tumor cells ex vivo to generate a population of cultured tumor cells, contacting population with a test therapeutic and a metabolic profiling compound comprising [1,2-¹³C₂]-D-glucose; and determining the ratio of [1-¹³C₁]-D-ribose to [1,2-¹³C₂]-D-ribose in the population. Generally, a ratio of [1-¹³C₁]-D-ribose to [1,2-¹³C₂]-D-ribose of 1 or higher indicates that the therapeutic is appropriate for treating the subject. Alternatively, a ratio of [1-¹³C₁]-D-ribose to [1,2-¹³C₂]-D-ribose of lower than 1 indicates that the therapeutic is inappropriate for treating the subject. In embodiments of the invention, the subject suffers from CML or a GIST. In some embodiments, the test therapeutic is not a tyrosine kinase inhibitor, e.g., the test therapeutic is a targeted drug against other kinases or signaling constructs.

In another aspect, the invention provides a method of determining the progression of cancer in a subject who is undergoing cancer treatment with a cancer therapeutic or who is expected to undergo cancer treatment with the cancer therapeutic, by administering to the subject a metabolic profiling compound comprising [1,2-¹³C₂]-D-glucose, obtaining from the subject a biological sample, determining the ratio of [1-¹³C₁]-D-ribose to [1,2-¹³C₂]-D-ribose in the biological sample, administering to the subject a cancer therapeutic, and repeating the preceding steps one or more times, whereby a decrease in the ratio of [1-¹³C₁]-D-ribose to [1,2-¹³C₂]-D-ribose in the biological sample following administration of the cancer therapeutic indicates that the subject has or is at risk of having a reduced response to treatment with the cancer therapeutic.

In embodiments of the invention, the ratio of [1-¹³C₁]-D-ribose to [1,2-¹³C₂]-D-ribose prior to administration of the test therapeutic is above 1 and the ratio of [1-¹³C₁]-D-ribose to [1,2-¹³C₂]-D-ribose following administration of the cancer therapeutic is below 1, e.g., the ratio of [1-¹³C₁]-D-ribose to [1,2-¹³C₂]-D-ribose following administration of the cancer therapeutic is below 0.8, or below 0.6, or below 0.4. In other embodiments of the invention, the biological sample is blood, a tumor biopsy, a tumor aspirate, a cultured tumor cell, or bone marrow. The step of determining the ratio of [1-¹³C₁]-D-ribose to [1,2-¹³C₂]-D-ribose is performed using, for example, GC-MS or NMR. Th subject may suffer from CML or a GIST, and the cancer therapeutic is a tyrosine kinase inhibitor, such as imatinib (Gleevec™). In other embodiments of the invention, the biological sample is in situ tumor. The step of determining the ratio of [1-¹³C₁]-D-ribose to [1,2-¹³C₂]-D-ribose is performed using nuclear magnetic imaging based on [1-¹³C₁]-D-ribose to [1,2-¹³C₂]-D-ribose signal strengths and the integrated peak area ratios provide biological assessment of the tumor and its expected response to a therapy.

In a further aspect, the invention provides a kit that contains a metabolic profiling compound comprising [1,2-¹³C₂]-D-glucose, means for obtaining from a subject a biological sample, and instructions for use. The biological sample is blood, a tumor biopsy, a tumor aspirate, a cultured tumor cell, or bone marrow. The kit may be for use with a human subject who is suffering from or is at risk of cancer. The kit may also contain reagents, tubes and procedures to purify, derivatize and analyze ribose ¹³C isotopomers for calculating the ratio of [1-¹³C₁]-D-ribose to [1,2-¹³C₂]-D-ribose in the biological sample. The kit may also contain GC-MS methods, temperature programming, retention times, sequence loading and instrument settings and peak integration parameters for the selective ion monitoring (SIM) of ribose isotopomers and for accurately measuring the ratio of [1-¹³C₁]-D-ribose to [1,2-¹³C₂]-D-ribose in the biological sample. The kit may also contain instructions and spreadsheet macros or a computer software for accurately subtracting natural ¹³C abundance and accurately calculating the ratio of [1-¹³C₁]-D-ribose to [1,2-¹³C₂]-D-ribose in the biological sample.

These and other objects, advantages, and features of the invention will become apparent to those persons skilled in the art upon reading the details of the invention as more fully described below. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

### BRIEF DESCRIPTION OF THE DRAWINGS

FIG. 1 depicts the chemical structures and processing of [1,2-¹³C₂]-D-glucose, a preferred metabolic profiling compound of the invention, and [1,2-¹³C₂]-D-glucose-6-phosphate, an activated (e.g., phosphorylated) metabolic profiling compound of the invention.

FIG. 2 depicts the use of [1,2-¹³C₂]-D-glucose in the production of [1-¹³C₁]-D-ribulose-5-phosphate in the oxidative branch of the pentose cycle. The first tracer carbon of glucose is lost in the form of ¹³CO₂, yielding [1-¹³C₁]-D-ribulose-5-phosphate.

FIG. 3 depicts the use of [1-¹³C₁]-D-ribulose-5-phosphate produced in the oxidative branch of the pentose cycle to yield [1-¹³C₁]-D-ribose-5-phosphate for nucleic acid synthesis. The mass spectral peak corresponding to [1-¹³C₁]-D-ribose is the second peak from the left and is also indicated by the solid arrow. The height of this peak corresponds to fractional oxidative ribose synthesis and is used in this patent to calculate drug resistance. Relative increase of this spectral peak indicates sensitivity to Gleevec in leukemia or other cancers. Mass per charge [m/z] of unlabeled derivatized ribose is 256 and the mass per charge [m/z] of ¹³C labeled derivatized ribose in the first carbon position is 257.

FIG. 4 depicts the use of [1,2-¹³C₂]-D-glucose in the production of [1,2-¹³C₂]-D-ribose-5-phosphate in the non-oxidative branch of the pentose cycle for nucleic acid synthesis. There is no net carbon loss via the non-oxidative steps of the pentose cycle, therefore ribose formed via these reactions retains both tracer carbons in the first and second positions. The mass spectral peak corresponding to [1,2-¹³C₂]-D-ribose is the third peak from the left and is also indicated by the solid black arrow. The height of this peak corresponds to fractional non-oxidative ribose synthesis and is used in this patent to calculate drug resistance. Relative increase of this spectral peak indicates developing drug resistance to Gleevec in leukemia and other cancers. Mass per charge [m/z] of unlabeled derivatized ribose is 256 and the mass per charge [m/z] of ¹³C labeled derivatized ribose in the first and second carbon positions is 258.

FIG. 5 depicts the results of studies comparing the ratio of [1-¹³C₁]-D-ribose to [1,2-¹³C₂]-D-ribose in cultured myeloid cells from patients with chronic myeloid leukemia using increasing concentrations of Gleevec (“x” axis). The set of vertical bars on the left are observed values of m1 and the set of vertical bars on the right are observed values of m2. The calculated ratios of m1/m2 are provided above the horizontal lines. In this experiment, a cut off point of 0.65 to detect resistance was used to identify Gleevec resistance. Cells from two patients resistant to Gleevec have m1/m2 ratios below 0.65, as indicated by an asterisk.

FIG. 6 depicts the results of in culture studies comparing the ratio of [1-¹³C₁]-D-ribose to [1,2-¹³C₂]-D-ribose in rat neuroblastoma (C6) cells which are sensitive to imatinib treatment. The increased ratio of m1/m2 ¹³C in ribose indicates sensitivity to Gleevec, which is further increased by Hydroxyurea treatment, which is a well known chemotherapeutic agent for the treatment of various human cancers. The set of vertical bars on the left are observed values of m1 and the set of vertical bars on the right are observed values of m2. The calculated ratios of m1/m2 are provided above the horizontal lines.

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

Definitions

For convenience, certain terms used in the specification, examples, and appended claims are collected here. Unless otherwise defined, all technical and scientific terms used herein have the same meaning as commonly understood by one of ordinary skill in the art to which this invention pertains. However, to the extent that these definitions vary from meanings circulating within the art, the definitions below are to control.

A “molecular profiling compound” includes any conjugates, substitutes or derivatives of the compound, produced by natural or synthetic means. Generally, a molecular profiling compound of the invention is fully and readily metabolized by any organism with no toxicity or side effects. A molecular profiling compound of the invention is indistinguishable from the natural substrate (e.g., glucose) by taste, color, solubility or any chemical/physical properties other than mass (molecular weight) and response to an electromagnetic field and energies of radio frequencies.

A “metabolite” includes any molecule produced by any metabolic process.

“Subject” includes living organisms such as humans, monkeys, cows, sheep, horses, pigs, cattle, goats, dogs, cats, mice, rats, cultured cells therefrom, and transgenic species thereof. In a preferred embodiment, the subject is a human. A subject is synonymous with a “patient.” Administration of the compositions of the present invention to a subject to be treated can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced as indicated by the exigencies of the therapeutic situation.

“Substantially pure” includes compounds, e.g., drugs, proteins or polypeptides that have been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state. Included within the meaning of the term “substantially pure” are compounds, such as proteins or polypeptides, which are homogeneously pure, for example, where at least 95% of the total protein (by volume, by wet or dry weight, or by mole percent or mole fraction) in a sample is the protein or polypeptide of interest.

“Administering” includes routes of administration which allow the compositions of the invention to perform their intended function, e.g., treating or preventing cardiac injury caused by hypoxia or ischemia. A variety of routes of administration are possible including, but not necessarily limited to parenteral (e.g., intravenous, intraarterial, intramuscular, subcutaneous injection), oral (e.g., dietary), topical, nasal, rectal, or via slow releasing microcarriers depending on the disease or condition to be treated. Oral, parenteral and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980)).

“Effective amount” includes those amounts of the compositions of the invention which allow it to perform its intended function, e.g., treating or preventing, partially or totally, cancer or other celluloproliferative disease, as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg/day to about 500 mg/kg/day, advantageously from about 0.1 mg/kg/day to about 100 mg/kg/day. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Pharmaceutically acceptable esters” includes relatively non-toxic, esterified products of therapeutic compounds of the invention. These esters can be prepared in situ during the final isolation and purification of the therapeutic compounds or by separately reacting the purified therapeutic compound in its free acid form or hydroxyl with a suitable esterifying agent; either of which are methods known to those skilled in the art. Acids can be converted into esters according to methods well known to one of ordinary skill in the art, e.g., via treatment with an alcohol in the presence of a catalyst.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, e.g., in Remington's Pharmaceutical Sciences.

Methods and Compositions for Analyzing Metabolic Pathways in Cancer

The present invention is based, in part, on the ability to differentiate biological products (such as metabolites) that are produced by different cellular pathways using labeled compounds used in these pathways, which are termed molecular profiling compounds. This differentiation allows the prediction of drug response or resistance of a tumor as well as a more detailed diagnosis of a subject's cancer than was previously available to the clinician. The present invention is based, in part, on at least two independent metabolic network reactions producing the same metabolite for nucleic acid backbone sugar synthesis and the contributions of these two reactions to the metabolite pool as the biomarker of disease progression and drug response in cancer.

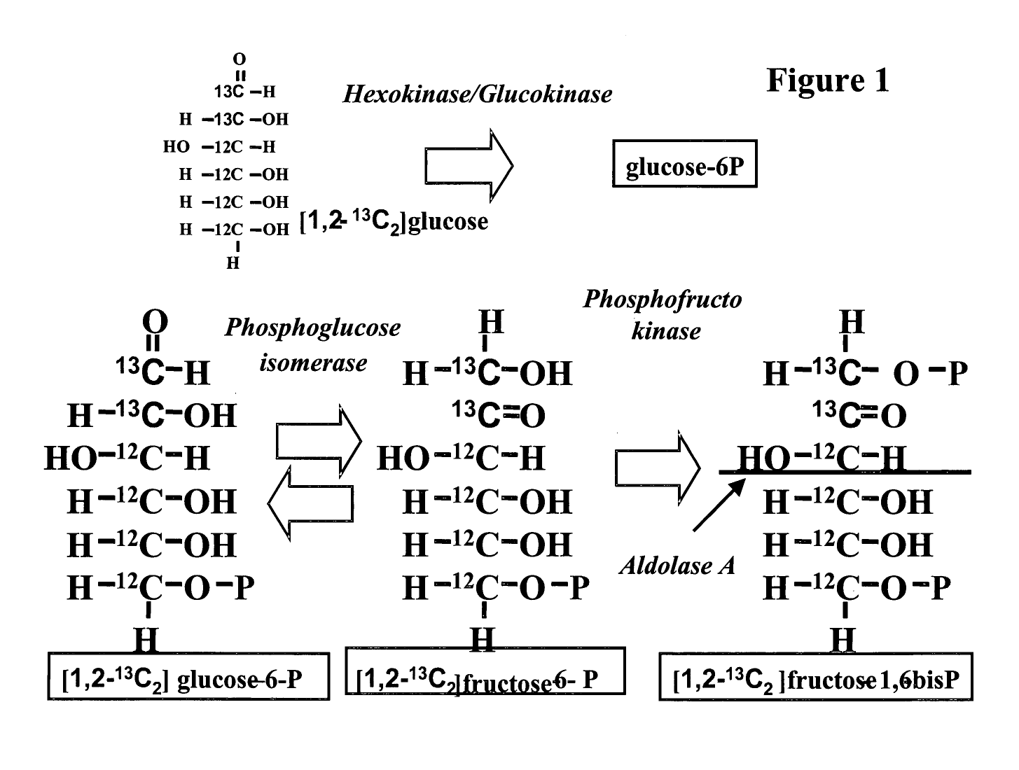

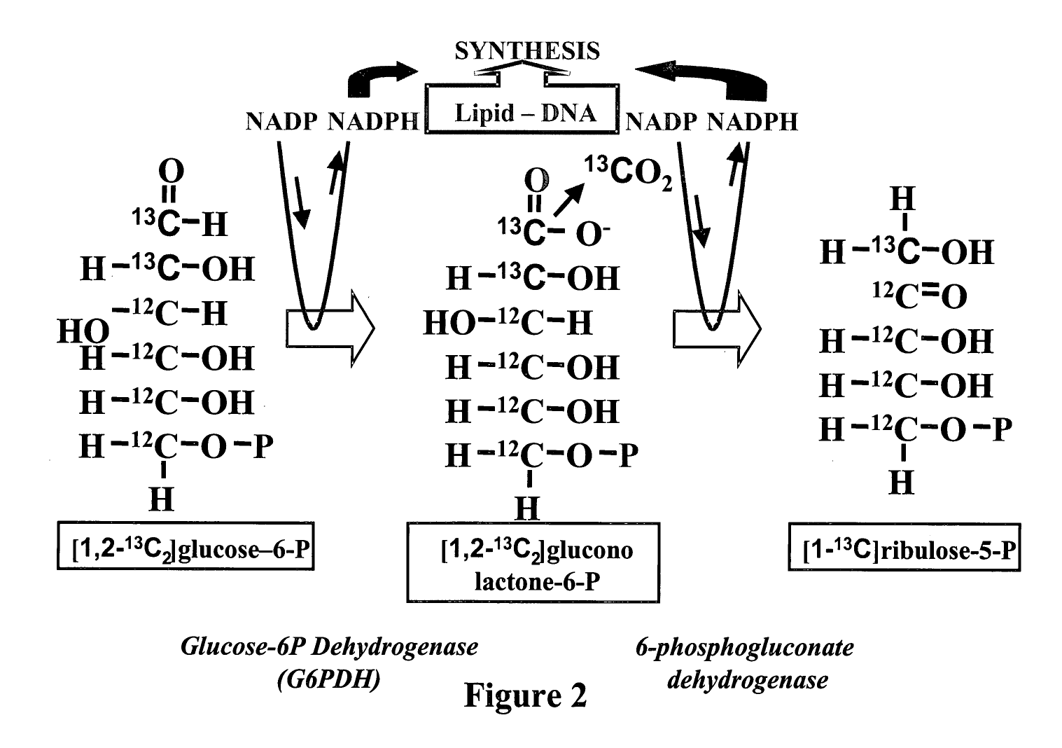

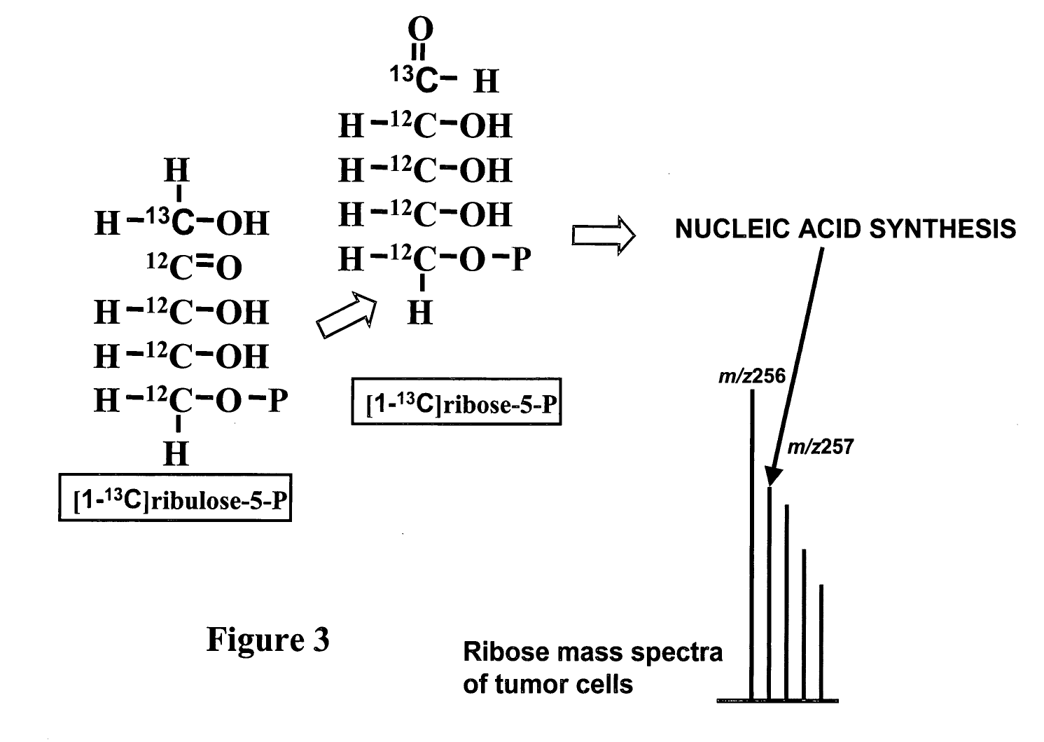

The stable ¹³C isotope-based glucose substrates [1,2-¹³C₂]-glucose and [1,2-¹³C₂]-D-glucose-6-phosphate (See FIG. 1) readily and dynamically label intracellular metabolic pathways through active metabolic steps resulting in stable, isotope labeled products, including ribose and deoxyribose. Glucose phosphorylation occurs by hexokinase or glucokinase activities before glucose is broken down into three carbon metabolites via anaerobic glycolysis. All human cells, including cancer cells, possess two major pathways to produce nucleic acid precursors: i) the oxidative and ii) the non-oxidative route of the pentose cycle. (See FIGS. 2-4). Use of the [1,2-¹³C₂]-glucose metabolic profiling compound in the oxidative pathway of the pentose cycle results in the production of [1-¹³C₁]-D-ribose or [1-¹³C₁]-D-deoxyribose. Conversely, use of the [1,2-¹³C₂]-D-glucose metabolic profiling compound in the non-oxidative pathway of the pentose cycle results in the production of [1,2-¹³C₂]-D-ribose or [1,2-¹³C₂]-D-deoxyribose. The usage of these metabolic pathways is perturbed in cancer, particularly when the cancer is becoming resistant to a drug treatment. Thus, the ratio of oxidative to non-oxidative sugar phosphate synthesis for nucleic acids (i.e., RNA and DNA) provides a means for determining the likelihood of partial or complete drug resistance in a patient undergoing a cancer treatment.

Generation of Molecular Profiling Compounds

The method of the invention can be carried out with a number of molecular profiling compounds. The molecular profiling compounds are preferably molecules labeled with ¹³C isotope at a known position, which are provided in the form of a precursor molecule that is converted to a quantifiable metabolite and its ¹³C labeled derivatives on various carbon position, known as mass isotopomers. The precursor molecule can be any molecule which normally contains a ¹²C. Further, 1, 2, 3, 4, 5, 6 or any number of ¹³C labels can be included within the precursor molecule. An example of a precursor molecule typically utilized in connection with the invention is a D-glucose molecule. Glucose enters the cell as a broad and widely used substrate, or precursor of others, upon which it becomes activated (phosphorylated) by the enzymes hexokinase or glucokinase. Glucose provides carbons for the synthesis of glycogen, pentoses, nucleotides, glycolysis intermediates, TCA cycle metabolites, fatty acids, lactate, amino acids and many other molecules not discussed here. A preferred molecular profiling compound is [1,2-¹³C₂]-D-glucose. Stable [1,2-¹³C₂]-D-glucose preferably is >99% purity and >99% isotope enrichment for each position, and is commercially available. (Isotec, Inc., Miamisburg, Ohio; Cambridge Isotope Laboratories, Andover, Mass.; Spectra Stable Isotopes, Columbia, Md.). Other molecular profiling compounds include [1,2,5,6-¹³C₄]-D-glucose and [U-¹³C₁₈]-stearic acid.

The changing pattern of distribution of ¹³C carbons from [1,2-¹³C₂]-D-glucose in intracellular metabolic intermediates may be used to provide a measure of carbon flow toward the pentose cycle using either the oxidative or the non-oxidative branches. Conventional label systems generally do not differentiate between the two branches, which produce chemically identical ribose. By the rearrangements of ¹³C in ribose the method of the invention can be used to differentiate between oxidative and non-oxidative pentose production.

In general, [1,2-¹³C₂]-D-glucose metabolism produces four isotope-labeled intermediary metabolite species, also called mass isotopomers, m1: with one ¹³C substitution; m2: with two ¹³C substitutions; m3: with three ¹³C substitutions; and m4: with four ¹³C substitutions; which can reside in various positions in intermediary metabolites. Without wishing to be limited by theory, it is generally believed that there is no known metabolic reaction or combination of reactions which produces m5: ribose with five ¹³C substitutions. These isotopomers are readily separated and measured using gas chromatography/mass spectrometry or NMR techniques described below.

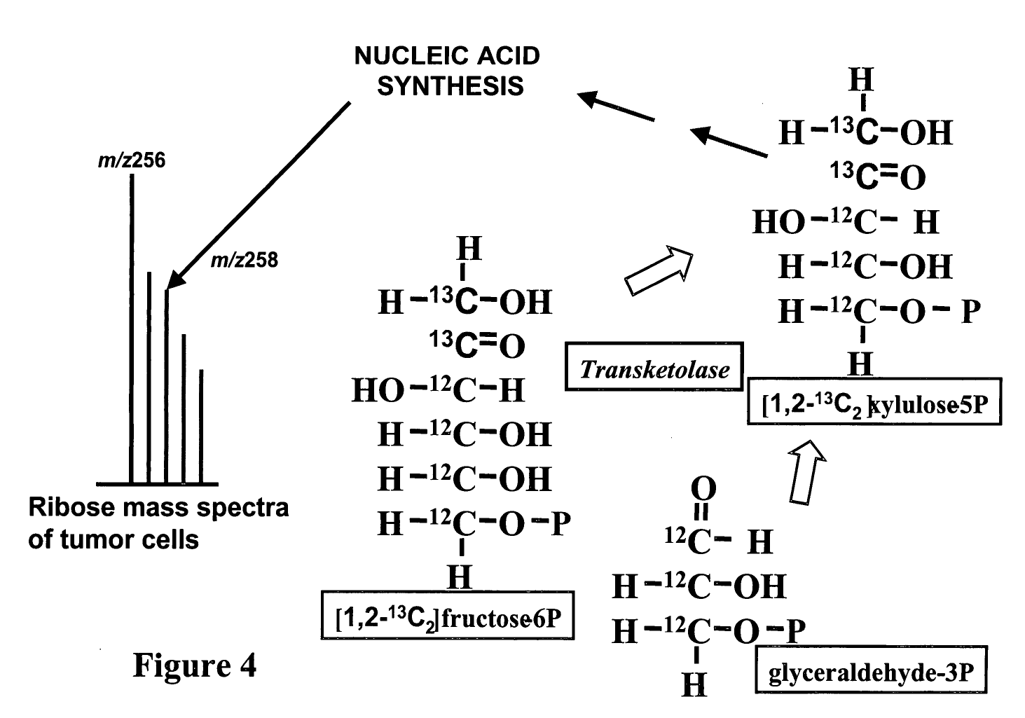

Ribose and deoxyribose are the building blocks of nucleotides and therefore ¹³C incorporation from glucose into RNA ribose or DNA deoxyribose indicates changes in nucleic acid synthesis rates through the respective branches of the pentose cycle. Singly labeled ribose molecules on the first carbon position (m1) represent ribose that is produced by direct glucose oxidation through glucose-6 phosphate dehydrogenase (G6PDH) and 6-phosphogluconate dehydrogenase (FIG. 2). This ribose can either be incorporated into nucleic acid or returned to glycolysis. The alternative pathway for ribose synthesis is through the non-oxidative steps of the pentose cycle using glycolytic metabolites (FIG. 4). There is no net carbon loss throughout the non-oxidative steps of the pentose cycle; therefore, ribose molecules labeled on the first two carbon positions with ¹³C (m2) represent nucleic acid ribose synthesis through the non-oxidative route. The ratio between m1, m2, m3 and m4 of nucleic acid ribose/deoxyribose closely reflects the involvement of glucose oxidation and non-oxidative ribose synthesis in tumor cells that become partially or fully drug-resistant. The targeted drug imatinib (Gleevec™) primarily controls oxidative ribose synthesis in the pentose cycle, therefore, if the non-oxidative route is utilized by tumor cells, manifested by a decrease in the [(m1/Σm)/(m2/Σm)] ratio, resistance to the drug is determined.

Additionally, other metabolic patterns including glycolysis, direct glucose oxidation, TCA cycle and fatty acid synthesis, may be examined in order to reveal specific changes in lactate, glutamate, palmitate and CO₂ during disease and health or during drug treatments or other interventions.

Lactate is the main three-carbon product of glycolysis and it is readily secreted into the cell culture medium. Accordingly, lactate can be utilized for the measurement of label incorporation into the three-carbon metabolite pool.

Those skilled in the art reading this disclosure will recognize that the ratio between m1 (recycled lactate from oxidized glucose via the oxidative branch of pentose cycle) and m2 (lactate produced by the Embden-Meyerhof-Parnas glycolytic pathway) is indicative of the activity of G6PDH and glucose recycling in the pentose cycle. A detailed description of the reactions and calculations can be found elsewhere (Lee, W. N., Boros, L. G., Puigjaner, J., Bassilian, S., Lim, S., Cascante, M. (1998) Mass isotopomer study of the non-oxidative pathways of the pentose cycle with [1,2-¹³C₂]glucose. Am. J. Physiol. 274, E843-51). Disease processes and drug treatments that affects direct glucose oxidation or glycolytic flux is expected to alter glucose label rearrangement in lactate.

¹³CO₂ release is a reliable marker of glucose oxidation. ¹³CO₂ production from [1,2-¹³C₂]-D-glucose takes place in both the pentose and TCA cycles and it is measured as part of the metabolic profiling processes to determine the rate of glucose oxidation in response to various drug therapies. Decreased glucose oxidation with increased glucose uptake is always a reliable sign of increased anabolism as seen in transformed cells.

Glutamate, a non-essential amino acid, is partially produced from mitochondrial α-ketoglutarate, which is a central intermediate of the TCA cycle. Glutamate is readily released into the culture medium after synthesis, which represents one of the routes for glucose carbon utilization. Therefore, label incorporation from glucose into glutamate is a good indicator of TCA cycle anabolic metabolism for amino acid synthesis instead of glucose oxidation.

Fatty acid synthesis is also strongly dependent on glucose carbons through the formation of acetyl-CoA via pyruvate dehydrogenase. The incorporation of ¹³C from [1,2-¹³C₂]-D-glucose gives key information about the fraction of de novo lipogenesis in mammalian cells and about glucose carbon contribution to acetyl-CoA for fatty acid synthesis. Many diseases and treatment modalities alter fatty acid synthesis, and changes in the flow of carbon toward fatty acid synthesis are important in cell growth control, differentiation, enzyme/hormone synthesis and new receptor formation.

Additional processes that further constitute the network are fatty acid chain elongation, chain desaturation and fatty acid β-oxidation, which can also be quantitatively determined using the metabolic tracer described in this invention. Furthermore, fatty acid chain shortening either via peroxysomal or mitochondrial β-oxidation can be determined and carbon flow measured via glyconeogenesis (reverse flux) can be quantified. Metabolic processes and their hierarchies are linked via co-factors such as NADP and NADP⁺, therefore control points and control properties of metabolic enzymes are also determined using the methods of the invention.

Detection of Labeled Products of Profiling Compounds

Separation of the [1-¹³C₁]-D-ribose or [1-¹³C₁]-D-deoxyribose produced by the oxidative branch of the pentose cycle and the [1,2-¹³C₂]-D-ribose or [1,2-¹³C₂]-deoxyribose produced in the non-oxidative branch of the pentose cycle is performed using any of a multitude of detection methods as described below. (See, Andrew, R. (2001) Clinical measurement of steroid metabolism. Best Pract Res Clin Endocrinol Metab 15: 1-16; Lee, W. N., Boros, L. G., Puigjaner, J., Bassilian, S., Lim, S., Cascante, M. (1998) Mass isotopomer study of the non-oxidative pathways of the pentose cycle with [1,2-¹³C₂]glucose. Am. J. Physiol. 274, E843-51; Lee, W. N., Edmond, J., Bassilian, S., Morrow, J. W. (1996) Mass isotopomer study of glutamine oxidation and synthesis in primary culture of astrocytes. Dev. Neurosci. 18, 469-77; Lee, W. N., Byerley, L. O., Bassilian, S., Ajie, H. O., Clark, I., Edmond, J., Bergner, E. A. (1995) Isotopomer study of lipogenesis in human hepatoma cells in culture: contribution of carbon and hydrogen atoms from glucose. Anal. Biochem. 226, 100-12).

Mass Spectroscopy (MS) Detectors

The sample compound or molecule is ionized, it is passed through a mass analyzer, and the ion current is detected. There are various methods for ionization. Types of ion sources include electrospray ionization, chemical ionization, fast atom bombardment, matrix-assisted laser desorption ionization, Thermal ionization (TIMS), Secondary ionization (SIMS), Selected Ion Flow Tube and Plasma source. The mass analyzer uses the kinetic energy imparted by motion through an electric field. This gives the particles an inertia dependent on the particle's mass to steer certain masses to the detector based on their mass-to-charge ratios (m/z) by varying the electrical or magnetic field, or both. Without wishing to be limited by theory, it is generally believed that the separation of masses follows the classic laws of physics according to the second law of motion by Isaac Newton [a=F/m]. First, this law states that if a force is exerted on an object, it will accelerate, i.e., it changes its velocity, and it will change its velocity in the direction of the force. Second, this acceleration is directly proportional to the force. Third, this acceleration is inversely proportional to the mass of the object. In the invention, all laws of motion are applied and obeyed, i.e., the metabolite moves in the direction of a repelling force in the ionization source. Therefore, a ribose molecule that is labeled on the first or the first and second positions with the heavy ¹³C tracer placed in a horizontally constant electromagnetic field will change velocity at different rates. The inventors presently demonstrate that because the oxidative and non-oxidative biochemical reactions of the pentose cycle rearrange the heavy carbons of [1,2-¹³C₂]-D-glucose into specific positions in ribose and deoxyribose, mass spectra obtained from subjects, e.g., subjects affected by or at risk from a disease or subjects undergoing drug treatment, reveal metabolic pathway distinctions, such as abnormalities and drug resistance. The present invention provides methods of identifying these pathway distinctions, by generating ratios of the two major isotopomers of ribose and deoxyribose, i.e., by denominating one with the other, and thereby provides intracellular metabolic network flow information in physiology and pathology as well as during drug treatment within a dynamic system.

The mass analyzer can be used to select a narrow range of m/z or to scan through a range of m/z to catalog the ions present. Mass analyzers include magnetic-sector analyzers as well as time-of-flight, ion trap, and quadrupole mass analyzers. The invention also provides for tandem mass spectrometers, which are capable of multiple rounds of mass spectrometry, such as for use in called collision induced dissociation analyses. For the invention a quadrupole mass analyzer is provided as a non-limiting example of exercising the art as herein described. Other instruments applying the same principles described herein are known to those skilled in the art. Other non-limiting examples are also described.

Pyrolysis Mass Spectrometry

Pyrolysis is the thermal degradation of complex material in an inert atmosphere or vacuum. It causes molecules to cleave at their weakest points to produce smaller, volatile fragments called pyrolysate (Irwin 1982). Curie-point pyrolysis is a particularly reproducible and straightforward version of the technique, in which the sample, dried onto an appropriate metal is rapidly heated to the Curie-point of the metal. A mass spectrometer can then be used to separate the components of the pyrolysate on the basis of their mass-to-charge ratio to produce a pyrolysis mass spectrum (Meuzelaar et al 1982) which can then be used as a “chemical profile” or fingerprint of the complex material analyzed. The combined technique is known as pyrolysis mass spectrometry (PyMS).

Nuclear Magnetic Resonance (NMR) Detectors

Certain nuclei with odd-numbered masses, including ¹³C and H, spin about an axis in a random fashion. When they are placed between poles of a strong magnet, the spins are aligned either parallel or anti-parallel to the magnetic field, with parallel orientation favored since it is slightly lower energy. The nuclei are then irradiated with electromagnetic radiation which is absorbed and places the parallel nuclei into a higher energy state where they become in resonance with radiation. Different spectra will be produced depending on the location of the H or ¹³C and on adjacent molecules or elements in the compound because all nuclei in molecules are surrounded by electron clouds which change the encompassing magnetic field and thereby alter the absorption frequency. For any NMR analysis, the signal-to-noise ratio (S/N) can be improved by signal averaging. Signal averaging increases S/N by the square-root of the number of signals taken. The invention provides for the use of Fourier transform NMR spectroscopy (FT-NMR) which decreases the time it takes to acquire a scan by allowing a range of frequencies to be probed at once. The invention provides for the use of a computer capable of performing the computationally-intensive mathematical transformation of the data from the time domain to the frequency domain, to produce a spectrum. The invention provides for the use of [1-¹³C₁]-D-glucose as the NMR metabolic tracer, where the accumulation of [1-¹³C₁]-D-ribose and [1-¹³C₁]-deoxyribose are monitored as indicators of an increase in non-oxidative nucleic acid sugar precursor synthesis. The invention further provides the use of multi-dimensional nuclear magnetic resonance spectroscopy, in which there are at least two pulses, and as the experiment is repeated, the time between a pair of pulses is varied. The first dimension is the frequency of the excitation, and the second dimension is based on the time differential between the pair of pulses (because of the properties of the Fourier transform, this second dimension is eventually expressed as a frequency as well). In multidimensional nuclear magnetic resonance, there will be a sequence of pulses, and at least one variable time period (in 3D, two time sequences will be varied. In 4D, three will be varied).

Fourier Transform Infrared Spectroscopy (FT-IR)

This method measures dominantly vibrations of functional groups and highly polar bonds. The generated fingerprints are made up of the vibrational features of all the sample components (Griffiths 1986). FT-IR spectrometers record the interaction of IR radiation with experimental samples, measuring the frequencies at which the sample absorbs the radiation and the intensities of the absorptions. Determining these frequencies allows identification of the samples chemical makeup, since chemical functional groups are known to absorb light at specific frequencies. Both quantitative and qualitative analysis are possible using the FT-IR detection method.

Sample Procurement and Preparation

The present invention provides biological samples from which the ratio of [1-¹³C₁]-D-ribose to [1,2-¹³C₂]-D-ribose is determined. As used herein, a “biological sample” includes any organic material obtained from a subject, from which a ratio of labeled metabolites can be determined. In preferred embodiments, the biological sample is blood, urine, a tumor biopsy, a tumor aspirate, a cultured tumor cell, or bone marrow. Biological samples are typically obtained by standard methods of blood drawing through a needle, collecting urine in a container, needle biopsy during minor surgery or explorative surgery by visual or ultrasound-guided approaches, standard culturing methods (e.g., techniques using a laminar sterile airflow biosafety cabinet or hood), by sternal puncture, or by puncture of the pelvic bone.

In embodiments of the invention, different fractionation procedures can be used to enrich the biological samples for small specific molecules. The molecules obtained are then passed over one or several fractionation columns.

Methods for Determining the Likelihood of a Subject's Reduced Response to Treatment with a Cancer Therapeutic

Drug therapy is a common strategy for treating a subject suffering from cancer. However, cells within the tumors become drug-resistant, decreasing the efficacy of the treatment. For example, imatinib (also known as STI-571, Glivec™ or Gleevec™) is used to treat Philadelphia chromosome positive chronic myeloid leukemia (CML) and gastrointestinal stromal tumors (GISTs), but subjects treated with imatinib often have a reduced response to the drug, (the tumor becomes “drug-resistant”), meaning that a given dosage of imatinib does not result in tumor regression or stasis. A reduced response includes any measurable reduction in response, while a fully-resistant tumor is one in which imatinib treatment at normal levels does not provide a detectable improvement in the subject's condition. Generally, resistance to imatinib is accompanied by a blast crisis with an average survival of 4.5 months.

The present invention provides methods for determining the likelihood of a subject's reduced response to treatment with a cancer therapeutic, such as imatinib or any other targeted kinase inhibitor, signal transduction inhibitor (STI) or gene modulator. A subject may be suspected of having a drug resistant cancer based on duration of treatment, lack of tumor regression, or other clinical symptoms. Generally, drug resistance is initially determined or confirmed by one or more clinical tests. The most common “test” is the lack of response to increased doses of the drug in the patient. To date there are no effective methods to detect early drug resistance before clinical drug resistance occurs. In leukemia, drug resistance is revealed by an increasing number of poorly differentiated blasts and their progenies. Molecular markers confirm drug resistance by detecting a mutated target protein, high expression of a target gene or the loss of a target protein drug binding domain or its signaling component. As yet, these molecular markers have limited predictive value and specificity to reveal outcome. Multiple protein mutations and losses of drug binding domains increase the possible permutations in the mechanisms of drug resistance against targeted gene constructs and their protein products to multiple magnitudes. Alternatively, a subject may not have a drug-resistant tumor, but is advantageous to determine the likelihood that the subject will develop at any point a reduced response to treatment with a cancer therapeutic. It is advantageous to detect drug resistance as early as possible, such as in the early clonal stage, for a successful management of a disease involving pathologic cell proliferation (a celluloproliferative disease), such as cancer. Other celluloproliferative diseases include autoimmune diseases, chronic inflammation (e.g., arthritis), increased tissue repair (e.g., keloids), tissue reconstruction (e.g., cirrhosis) and pathologic tissue regeneration (or wasting).

The methods of the invention are routinely used during the management of cancer when progression of the disease state is evaluated. It is not necessary that the subject is identified as one who has or is at risk of having a drug-resistant tumor. A metabolic profiling compound comprising [1,2-¹³C₂]-D-glucose is administered to the subject, e.g., orally, at a dose of 1 gram/kilogram (g/kg) body weight, such that the [1,2-¹³C₂]-D-glucose enters a tumor cell of the subject. Generally, any method of administration now known or foreseeable in the future can be used to deliver the metabolic profiling compound. By way of non-limiting example, the metabolic profiling compound is administered to the subject intravenously at a dosage of about 1 g/kg of the subject's body weight. It is understood that this dose of the metabolic profiling compound [1,2-¹³C₂]-D-glucose provides about 25% of tracer-enriched glucose after about sixty minutes of oral consumption in the plasma.

Following a sufficient period of time (e.g., ninety minutes) after administration to allow the metabolic profiling compound to enter a tumor cell and be metabolized (termed the “absorption phase or peak clamp”), a biological sample is obtained from the subject. For example, a period of time of about 1.5 hours following administration of the metabolic profiling compound elapses prior to obtaining the biological sample. Alternatively, the metabolism time is about 12, 9, 6, 3, 2, or 1 hours, or 45, 30, 15, 10, 5 or 1 minutes.

The biological sample is blood, urine, a tumor biopsy, a tumor aspirate, a cultured tumor cell, or a sample from the subject's bone marrow, and is obtained by methods well-known in the art. Generally, at least 5 mg of biological sample (by weight) is obtained for use in the methods of the invention. Preferably, a blood and/or urine sample and tumor biopsy sample are obtained from the subject.

Molecules containing the ¹³C label that are present in the biological sample are analyzed. Typically, the molecules containing the ¹³C label are measured by GC-MS or NMR, as described above. The molecules which are analyzed may be completely different molecules from the originally labeled ¹³C precursor molecules. For example, when the administered metabolic profiling compound is [1,2-¹³C₂]-D-glucose, molecules containing the ¹³C label include [1-¹³C₁]-D-ribose, [1,2-¹³C₂]-D-ribose, [1-¹³C₁]-D-deoxyribose, [1,2-¹³C₂]-D-deoxyribose, ¹³CO₂, and [2,3-¹³C₂]-DL-lactate. In embodiments of the invention, the molecules containing the ¹³C label that are measured are [1-¹³C₁]-D-ribose and [1,2-¹³C₂]-D-ribose. In other embodiments of the invention, the molecules containing the ¹³C label that are measured are [1-¹³C₁]-D-ribose, [1-¹³C₁]-D-deoxyribose, [1,2-¹³C₂]-D-ribose, and [1,2-¹³C₂]-D-deoxyribose. In still other embodiments of the invention, the molecules containing the ¹³C label that are measured are [1-¹³C₁]-D-deoxyribose and [1,2-¹³C₂]-D-deoxyribose. Once the [1-¹³C₁]-D-ribose and [1,2-¹³C₂]-D-ribose present in the biological sample are measured, the ratio of [1-¹³C₁]-D-ribose to [1,2-¹³C₂]-D-ribose in the biological sample is determined.

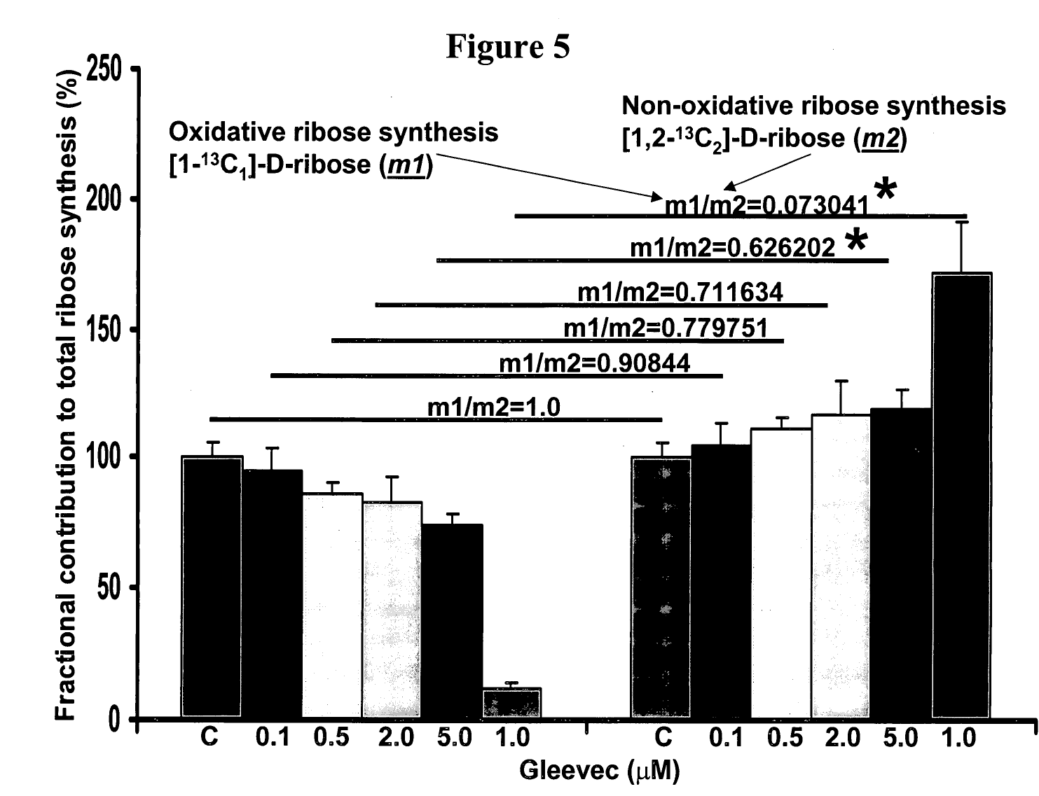

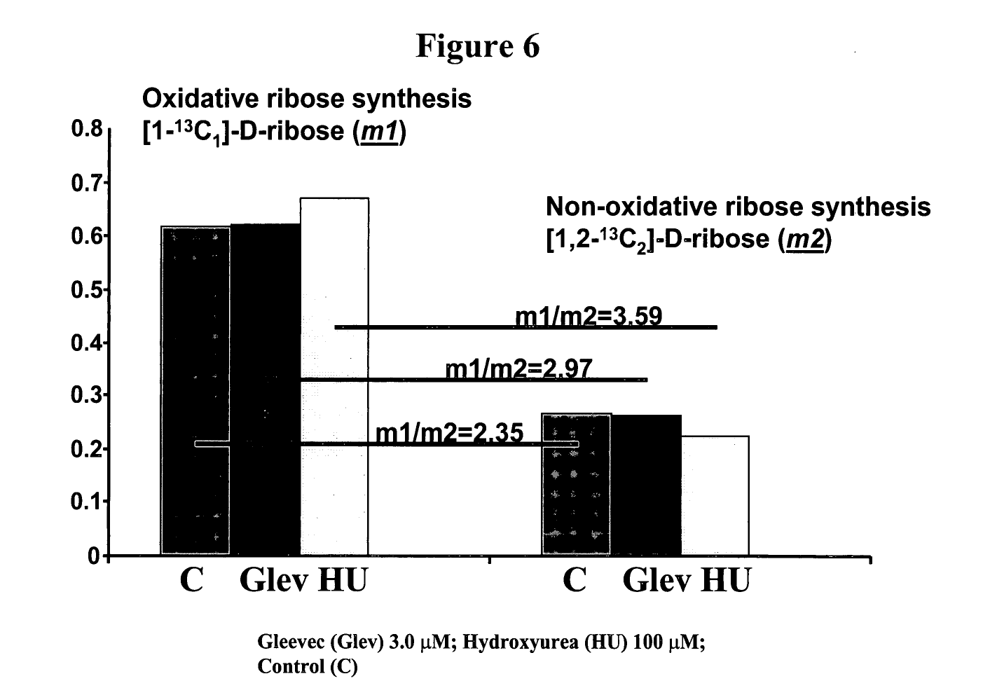

The present inventor has found that while normal cells use predominantly the oxidative branch of the pentose cycle in nucleic acid synthesis, which results in the production of [1-¹³C₁]-D-ribose or [1-¹³C₁]-deoxyribose from a [1,2-¹³C₂]-D-glucose molecule, tumor cells that are partially or fully drug-resistant increase the use of the non-oxidative branch of the pentose cycle of nucleic acid synthesis, which results in the production of [1,2-¹³C₂]-D-ribose or [1,2-¹³C₂]-deoxyribose from a [1,2-¹³C₂]-D-glucose molecule. Generally, a ratio of [1-¹³C₁]-D-ribose to [1,2-¹³C₂]-D-ribose of 1 or higher indicates that the subject is still responsive to imatinib treatment. When the ratio of [1-¹³C₁]-D-ribose to [1,2-¹³C₂]-D-ribose is less than 1 (e.g., 0.5) the patient is at risk of having a reduced response to treatment with a cancer therapeutic such as imatinib. The increased risk of developing a reduced response to imatinib includes any increased risk. In embodiments of the invention, the increased risk is about a 10% decrease in the ratio described above indicating the risk of developing imatinib resistance. In other embodiments, the increased risk is indicated by about a 10%, 15%, 20%, 25%, 50%, 75%, 85%, 90%, 95%, 98%, 99%, or 99.9% decrease in the ratio of [1-¹³C₁]-D-ribose to [1,2-¹³C₂]-D-ribose. In embodiments of the invention, the ratio of [1-¹³C₁]-D-ribose to [1,2-¹³C₂]-D-ribose at which a patient is diagnosed as having or being at risk of developing imatinib resistance is about 0.8, about 0.7, or about 0.6 or lower. As shown in FIG. 5, studies comparing the ratio of [1-¹³C₁]-D-ribose to [1,2-¹³C₂]-D-ribose in cultured myeloid cells from cancer patients (CML patients) undergoing imatinib treatment show that a ratio of [1-¹³C₁]-D-ribose to [1,2-¹³C₂]-D-ribose of 0.65 or lower indicates that the subject has imatinib resistance. In this experiment, a cut off point of 0.65 to detect resistance was used, as K562r and LAMA84r cells do not respond even to high doses of Gleevec treatment. Similarly, in culture studies were preformed to compare the ratio of [1-¹³C₁]-D-ribose to [1,2-¹³C₂]-D-ribose in rat neuroblastoma (C6) cells which are sensitive to imatinib treatment (See FIG. 6). The increased ratio of m1/m2 ¹³C in ribose (from 2.35 to 2.97 following Gleevec treatment) indicates sensitivity to Gleevec (i.e., the cells are not Gleevec-resistant), which is further increased (to 3.59) by hydroxyurea treatment, which is a well known chemotherapeutic agent for the treatment of human cancers.

Methods for Selecting an Appropriate Therapeutic for Treatment in a Subject Suffering from Cancer

Following diagnosis of a subject having a drug-resistant tumor, it is imperative to select an appropriate replacement therapeutic. Further, drug-resistant tumor cells may alter one or more metabolic processes so as to make them more sensitive to another class of inhibitory compounds. Similarly, it may be desirable to establish an alternative treatment regimen in a subject predicted to develop resistance to an anti-cancer drug.

The methods of the present invention can also be performed ex vivo by analyzing cells derived from a subject, such as tumor or cancer cells or stromal cells associated with the tumor or cancer (collectively termed “tumor cells”), in order to select such a replacement therapeutic. In embodiments of the invention, the cells are derived from a subject suffering from CML or a GIST. In embodiments of the invention, the cells are derived from a mammalian subject suffering from a neuroblastoma type of brain cancer. The test therapeutic is not a signal transduction inhibitor, such as a tyrosine kinase inhibitor. For example, the test therapeutic is a chemotherapeutic agent such as hydroxyurea. Alternatively, the test therapeutic is a tyrosine kinase inhibitor that targets a cellular pathway distinct from the pathway inhibited by imatinib, namely the Bcr-Abl signaling pathway. Further, the test therapeutic is a transketolase inhibitor. These test therapeutics may be c-KIT or platelet derived growth factor (PDGF) inhibitors. Another group of test therapeutics are direct metabolic enzyme inhibitors, such as oxythiamine for the inhibition of transketolase, which is a non-oxidative ribose synthesizing enzyme with a high flux control property in the cycle.

Generally the cells to be analyzed are removed from the subject in a biopsy procedure, such a needle biopsy. The cells are cultured using standard methods of cell culture known in the art, such that a population of cultured tumor cells is generated. The cultured cells are used directly as primary cells. Alternatively, the cultured cells are modified to generate, for example, stable cell lines. The primary cells or cell lines are contacted with a test therapeutic and a metabolic profiling compound that contains [1,2-¹³C₂]-D-glucose for a given period of time prior to generating a biological sample from the contacted cells. The population of cells is collected by means known in the art to provide a biological sample, such as by lysis in a detergent-containing solution, or by mechanical removal and disruption. For example, the period of time is of about 30 minutes following contact with the test therapeutic and the metabolic profiling compound. Alternatively, the metabolism time is about 6, 3, 2, or 1 hours, or 45, 15, 10, 5 or 1 minutes. Following sample collection, the ratio of [1-¹³C₁]-D-ribose to [1,2-¹³C₂]-D-ribose in the biological sample is determined essentially as described above. Generally, a [1-¹³C₁]-D-ribose to [1,2-¹³C₂]-D-ribose ratio of about 1 or higher (e.g., 1.5, 2, or greater) indicates that the test therapeutic is appropriate for treating the subject. Alternatively, the ratio of [1-¹³C₁]-D-ribose to [1,2-¹³C₂]-D-ribose is less than about 1 (e.g., 0.8, 0.7, 0.6 or lower), which indicates that the test therapeutic is inappropriate for treating the subject.

Methods for Monitoring the Progression of Cancer in a Subject who is Undergoing Cancer Treatment

It is useful for clinicians to determine accurately whether a cancer in a subject undergoing treatment is progressing and, if so, to what extent. The invention provides methods for monitoring cancer progression by monitoring the ratio of [1-¹³C₁]-D-ribose to [1,2-¹³C₂]-D-ribose produced by cells of the tumor or cancer.

A subject suffering from cancer who is at risk of cancer progression is identified. The subject is administered a metabolic profiling compound comprising [1,2-¹³C₂]-D-glucose. After a period of time, a biological sample is obtained from the subject and the ratio of [1-¹³C₁]-D-ribose to [1,2-¹³C₂]-D-ribose in the biological sample is determined. Subsequently, the subject is treated by administration of a cancer therapeutic. These steps are repeated one or more times, whereby a decrease in the ratio of [1-¹³C₁]-D-ribose to [1,2-¹³C₂]-D-ribose in the biological sample following administration of the cancer therapeutic indicates that the subject has or is at risk of having a reduced response to treatment with the cancer therapeutic. The decrease in the ratio of [1-¹³C₁]-D-ribose to [1,2-¹³C₂]-D-ribose may be quantified as either an absolute decrease or a threshold decrease. For example, the decrease in the ratio of [1-¹³C₁]-D-ribose to [1,2-¹³C₂]-D-ribose is over about 10%. Alternatively, the decrease in the ratio of [1-¹³C₁]-D-ribose to [1,2-¹³C₂]-D-ribose is over about 15%, 20%, 25%, 50%, 75%, or 100%. Alternatively, the ratio of [1-¹³C₁]-D-ribose to [1,2-¹³C₂]-D-ribose prior to administration of the test therapeutic is above a specific ratio, while the ratio of [1-¹³C₁]-D-ribose to [1,2-¹³C₂]-D-ribose following administration of the cancer therapeutic is below the specific ratio. For example, the ratio of [1-¹³C₁]-D-ribose to [1,2-¹³C₂]-D-ribose prior to administration of the test therapeutic is above 1 (e.g., 1.5, 2 or greater), while the ratio of [1-¹³C₁]-D-ribose to [1,2-¹³C₂]-D-ribose following administration of the cancer therapeutic is below 1 (e.g., 0.9, 0.8, 0.7, 0.6, 0.5 or less).

In embodiments of the invention, this method is performed prior to the subject receiving any treatment. Alternatively, this method can be initiated after the subject has received one or more treatments.

### EXAMPLES

### Example 1

### Correlation of Changes in the Ratio of [1-¹³C₁]-D-Ribose to [1,2-¹³C₂]-D-Ribose in Tumor Cells from CML Patients Undergoing Imatinib (Gleevec) Treatment

Myeloid (K-562) cells were obtained from peripheral blood of patients suffering from CML undergoing cancer therapy with Gleevec and cultured in vitro using standard culture conditions. These cultured CML myeloid cells were exposed to varying concentrations of Gleevec and contacted with [1,2-¹³C₂]-D-glucose. Samples of the treated cell cultures were isolated and subjected to ribose mass spectral analysis to obtain the ratio of [1-¹³C₁]-D-ribose to [1,2-¹³C₂]-D-ribose in the samples of the treated cell cultures. In this experiment, a cut off point of 0.65 or below to detect resistance was used, as it is known that K562r and LAMA84r cell lines do not respond well even to high doses of Gleevec treatment. The results of these studies are shown in FIG. 5. Myeloid cells from two patients have m1/m2 ratios below 0.65, as indicated by an asterisk, indicating that the subject has or is likely to develop imatinib resistance.

### Example 2

### Correlation of Changes in the ratio of [1-¹³C₁]-D-Ribose to [1,2-¹³C₂]-D-Ribose in Cultured Cell Lines Treated with Gleevec and Hydroxyurea

The ability of non-kinase inhibitor cancer therapeutics to alter the ratio of [1-¹³C₁]-D-ribose to [1,2-¹³C₂]-D-ribose in human cancers was investigated. Rat neuroblastoma (C6) cells are known to be sensitive to imatinib treatment; the ratio of [1-¹³C₁]-D-ribose to [1,2-¹³C₂]-D-ribose in cultured rat C6 neuroblastoma cells increases from 2.35 to 2.97 following treatment with 3.0 μM Gleevec. (See FIG. 6.) Cultured rat C6 neuroblastoma cells were incubated with 100 μM hydroxyurea. The ratio of [1-¹³C₁]-D-ribose to [1,2-¹³C₂]-D-ribose in hydroxyurea-treated cells increased (to 3.59) by hydroxyurea treatment.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of determining the likelihood of a subject's reduced response to treatment with a cancer therapeutic, comprising the steps of:
(a) administering to said subject a metabolic profiling compound comprising [1,2-¹³C₂]-D-glucose;
(b) obtaining from said subject a biological sample; and
(c) determining the ratio of [¹-¹³C₁]-D-ribose to [1,2-¹³C₂]-D-ribose in said biological sample; whereby a ratio below 1 indicates that said subject has or is at risk of having a reduced response to treatment with a cancer therapeutic.

2. The method of claim 1, wherein said cancer therapeutic is a tyrosine kinase inhibitor.

3. The method of claim 1, wherein said tyrosine kinase inhibitor is imatinib (Gleevec™).

4. The method of claim 1, wherein the ratio of [1-¹³C₁]-D-ribose to [1,2-¹³C₂]-D-ribose is 0.8 or lower.

5. The method of claim 1, wherein the ratio of [1-¹³C₁]-D-ribose to [1,2-¹³C₂]-D-ribose is 0.7 or lower.

6. The method of claim 1, wherein the ratio of [1-¹³C₁]-D-ribose to [1,2-¹³C₂]-D-ribose is 0.6 or lower.

7. The method of claim 1, wherein the ratio of [1-¹³C₁]-D-ribose to [1,2-¹³C₂]-D-ribose is 0.5 or lower.

8. The method of claim 1, wherein said biological sample is selected from the group consisting of blood, a tumor biopsy, a tumor aspirate, a cultured tumor cell, and bone marrow.

9. The method of claim 1, wherein the step of determining the ratio of [1-¹³C₁]-D-ribose to [1,2-¹³C₂]-D-ribose is performed using gas chromatography-mass spectroscopy (GC-MS) or nuclear magnetic resonance (NMR).

10. The method of claim 1, wherein said subject suffers from chronic myeloid leukemia (CML) or a gastrointestinal stromal tumor (GIST).

11. A method of selecting an appropriate therapeutic for treatment in a subject suffering from cancer who is partially or fully non-responsive to tyrosine kinase inhibitory treatment, comprising the steps of:
(a) obtaining from said subject one or more tumor cells;
(b) culturing said tumor cells ex vivo to generate a population of cultured tumor cells;
(c) contacting said population with a test therapeutic and a metabolic profiling compound comprising [1,2-¹³C₂]-D-glucose;
(d) determining the ratio of [1-¹³C₁]-D-ribose to [1,2-¹³C₂]-D-ribose in said population; whereby a ratio of 1 or higher indicates that said therapeutic is appropriate for treating said subject.

12. The method of claim 11, wherein the ratio of [1-¹³C₁]-D-ribose to [1,2-¹³C₂]-D-ribose is 1.5 or higher.

13. The method of claim 11, wherein the ratio of [1-¹³C₁]-D-ribose to [1,2-¹³C₂]-D-ribose is 2 or higher.

14. The method of claim 11, wherein said cancer is selected from the group consisting of chronic myeloid leukemia (CML) and a gastrointestinal stromal tumor (GIST).

15. The method of claim 1, wherein said therapeutic is not a tyrosine kinase inhibitor.

16. A method of determining the progression of cancer in a subject who is undergoing cancer treatment with a cancer therapeutic or is expected to undergo cancer treatment with said cancer therapeutic, comprising the steps of:
(a) administering to said subject a metabolic profiling compound comprising [1,2-¹³C₂]-D-glucose;
(b) obtaining from said subject a biological sample;
(c) determining the ratio of [1-¹³C₁]-D-ribose to [1,2-¹³C₂]-D-ribose in said biological sample;
(d) administering to said subject a cancer therapeutic;
(e) repeating steps (a) to (d) one or more times,
whereby a decrease in the ratio of [1-¹³C₁]-D-ribose to [1,2-¹³C₂]-D-ribose in said biological sample following administration of said cancer therapeutic indicates that said subject has or is at risk of having a reduced response to treatment with the cancer therapeutic.

17. The method of claim 16, wherein the ratio of [1-¹³C₁]-D-ribose to [1,2-¹³C₂]-D-ribose prior to administration of the test therapeutic is above 1 and the ratio of [1-¹³C₁]-D-ribose to [1,2-¹³C₂]-D-ribose following administration of the cancer therapeutic is below 1.

18. The method of claim 16, wherein the ratio of [1-¹³C₁]-D-ribose to [1,2-¹³C₂]-D-ribose prior to administration of the test therapeutic is above 1 and the ratio of [1-¹³C₁]-D-ribose to [1,2-¹³C₂]-D-ribose following administration of the cancer therapeutic is below 0.8.

19. The method of claim 16, wherein the ratio of [1-¹³C₁]-D-ribose to [1,2-¹³C₂]-D-ribose following administration of the cancer therapeutic is below 0.7.

20. The method of claim 16, wherein the ratio of [1-¹³C₁]-D-ribose to [1,2-¹³C₂]-D-ribose following administration of the cancer therapeutic is below 0.6.

21. The method of claim 16, wherein the ratio of [1-¹³C₁]-D-ribose to [1,2-¹³C₂]-D-ribose following administration of the cancer therapeutic is below 0.5.

22. The method of claim 16, wherein said biological sample is selected from the group consisting of blood, a tumor biopsy, a tumor aspirate, a cultured tumor cell, and bone marrow.

23. The method of claim 16, wherein the step of determining the ratio of [1-¹³C₁]-D-ribose to [1,2-¹³C₂]-D-ribose is performed using gas chromatography-mass spectroscopy (GC-MS) or nuclear magnetic resonance (NMR).

24. The method of claim 16, wherein said subject suffers from chronic myeloid leukemia (CML) or a gastrointestinal stromal tumor (GIST).

25. The method of claim 16, wherein said cancer therapeutic is a tyrosine kinase inhibitor.

26. The method of claim 25, wherein said tyrosine kinase inhibitor is imatinib (Gleevec™).

27. A kit comprising:
(a) a metabolic profiling compound comprising [1,2-¹³C₂]-D-glucose;
(b) means for obtaining from a subject a biological sample; and
(c) instructions for use thereof.

28. The kit of claim 27, wherein said biological sample is selected from the group consisting of blood, a tumor biopsy, a tumor aspirate, a cultured tumor cell, and bone marrow.

29. The kit of claim 27, wherein the subject is a human suffering from or is at risk of cancer.

30. The kit of claim 27, further comprising a means for calculating the ratio of [1-¹³C₁]-D-ribose to [1,2-¹³C₂]-D-ribose in said biological sample.

31. A method of identifying a subject having increased sensitivity to treatment with a cancer therapeutic that is not a tyrosine kinase inhibitor, comprising the steps of:
(a) administering to said subject a metabolic profiling compound comprising [1,2-¹³C₂]-D-glucose;
(b) obtaining from said subject a biological sample; and
(c) determining the ratio of [1-¹³C₁]-D-ribose to [1,2-¹³C₂]-D-ribose in said biological sample; whereby a ratio below 1 indicates that said subject has increased sensitivity to treatment with a cancer therapeutic that is not a tyrosine kinase inhibitor.

32. The method of claim 31, wherein said cancer therapeutic is a transketolase inhibitor.

33. A method of selecting an appropriate therapeutic for treatment in a subject suffering from cancer who is partially or fully non-responsive to a first inhibitor of a first metabolic pathway but is responsive to a second inhibitor of a second metabolic pathway, comprising the steps of:
(a) obtaining from said subject one or more tumor cells;
(b) culturing said tumor cells ex vivo to generate a population of cultured tumor cells;
(c) contacting said population with a test therapeutic and a metabolic profiling compound comprising [1,2-¹³C₂]-D-glucose;
(d) determining the ratio of [1-¹³C₁]-D-ribose to [1,2-¹³C₂]-D-ribose in said population; whereby a ratio of 1 or higher indicates that said therapeutic is an inhibitor of said second metabolic pathway and is appropriate for treating said subject.

34. The method of claim 33, wherein said first inhibitor is a tyrosine kinase inhibitor.

35. The method of claim 33, wherein said second inhibitor is a transketolase inhibitor.
